(19) United States
(12) Patent Application Publication
(10) Pub. No.: US 2008/0097492 A1
(43) Pub. Date: Apr. 24, 2008

(54) Vasuclar Graft Flange-Forming Apparatus and Method

(76) Inventor: William R. Hunter, Kingwood, TX (US)

Correspondence Address: QUICKPATENTS, INC., 32861 CALLE PERFECTO, SUITE A, SAN JUAN CAPISTRANO, CA, 92675, US

(21) Appl. No.: 11/551,662

(22) Filed: Oct. 20, 2006

(51) Int. Cl. A61B 17/08

(52) U.S. Cl. 606/153

(57) ABSTRACT

A hand tool and method of use for forming a flange on the end of a vascular graft is disclosed that includes a housing having a handle portion fixed to a trigger portion. A trigger is pivotally fixed within the housing at a lower side of the trigger portion, and includes a finger portion projecting downwardly from the housing. The trigger also includes an actuator portion, connected proximate a pivot that is connected itself to the finger portion. An anvil is pivotally fixed at a first end thereof within and to the trigger portion of the housing. Further, the anvil is slidably fixed at a second end to the actuator portion of the trigger. The anvil has a rigid top surface with a deformation portion thereon. A plurality of mandrels is adapted to laterally engage the deformation portion of the anvil, and each include a spring means for urging the mandrels towards the deformation portion of the anvil. A guide collar is fixed to an upper side of the trigger portion of the housing and has an aperture therein for receiving the open end of the graft and guiding same onto the deformation portion of the anvil. With the graft inserted into the aperture of the guide collar in such a fashion, the open end of the graft engages the deformation portion of the anvil. Manually pulling the finger portion of the trigger causes the actuator portion of the trigger to force the second end of the anvil upward, the mandrels holding the graft against the deformation portion of the anvil. As such, the deformation portion of the anvil causes the open end of the graft to deform into the flange.

### CROSS-REFERENCE TO RELATED APPLICATIONS

Not Applicable.

### STATEMENT REGARDING FEDERALLY SPONSORED RESEARCH AND DEVELOPMENT

Not Applicable.

### FIELD OF THE INVENTION

This invention relates to surgical devices, and more particularly to an apparatus for manually forming a flange on the end of a vascular graft.

### DISCUSSION OF RELATED ART

Vascular grafts having flanges formed into one end are common in the art of end-to-side anastomosis, and are used for bypassing a damaged section of a blood vessel, for example. Such grafts are pre-manufactured utilizing a variety of methods, such as, for example, those taught in U.S. Pat. No. 6,190,590 to Randall et al. on Feb. 20, 2001; U.S. Pat. No. 2,127,903 to Browen on Aug. 23, 1938; U.S. Pat. No. 4,909,979 to Possis et al. on Mar. 20, 1990; U.S. Pat. No. 4,957,508 to Kaneko et al. on Sep. 18, 1990; and U.S. Pat. No. 5,387,236 to Noishiki et al. on Feb. 7, 1995. Such manufacturing methods vary in their specific processes, but all have in common the necessity of pre-manufacturing the grafts outside of the operating room in which the grafts will be used. Such vascular grafts must be packaged in a sterile package after being manufactured in a clean room, and then shipped to the various hospitals offering such bypass procedures.

As such, a variety of sterile grafts of varying lengths and flange sizes must be available to the surgeon in the operating room, or accessible nearby by an attending nurse, so that once the surgeon determines the characteristics of the graft that is required in a particular instance it can be quickly secured. Clearly, however, there are considerable drawbacks to a system requiring a wide range of graft sizes of various materials, lengths, and flange sizes and shapes to be on-hand when operating on a patient. Not only may it be difficult to quickly find the appropriate graft required by the surgeon, but the optimal graft may not be available and a less desirable graft may be selected due to the constraints of time experienced while conducting open heart surgery.

Therefore, there is clearly a need for a device that allows a surgeon to form a flange of appropriate size and shape into the end of a vascular graft while in the operating room, during a surgical procedure. Such a needed device would allow the surgeon to form the proper flange in the open end of a vascular graft cut to the appropriate length, whether the graft is made from synthetic or natural materials, and with a tool that can be operated with one hand while holding the graft with the other hand. Such a needed invention would allow various shaped flanges to be selected, and the size of such flanges could be readily controlled by the user. Such a needed device would could be made to either be autoclaved or disposed of after the procedure. The present invention accomplishes these objectives.

### SUMMARY OF THE INVENTION

The present device is a hand tool for forming a flange on the end of a vascular graft. A user of such a device may grasp the hand tool with one hand, insert the graft into the tool, and form the flange, all within an operating environment for example. The hand tool includes a housing having a handle portion fixed to a trigger portion. A trigger is pivotally fixed within the housing at a lower side of the trigger portion, and includes a finger portion projecting downwardly from the housing. The trigger also includes an actuator portion, connected proximate a pivot that is connected itself to the finger portion. As such, when the finger portion is pulled towards the handle portion of the housing, the trigger pivots around the pivot to cause the actuator portion to lift within the housing. The finger portion is adapted to be actuated by a forefinger of the human hand while the hand is grasping the handle portion of the housing.

An anvil is pivotally fixed at a first end thereof within and to the trigger portion of the housing. Further, the anvil is slidably fixed at a second end to the actuator portion of the trigger. The anvil has a rigid top surface with a deformation portion thereon.

A plurality of mandrels is adapted to laterally engage the deformation portion of the anvil, and each include a spring means for urging the mandrels towards the deformation portion of the anvil.

A guide collar is fixed to an upper side of the trigger portion of the housing and has an aperture therein for receiving the open end of the graft and guiding same onto the deformation portion of the anvil. With the graft inserted into the aperture of the guide collar in such a fashion, the open end of the graft engages the deformation portion of the anvil. At this point, manually pulling the finger portion of the trigger causes the actuator portion of the trigger to force the second end of the anvil upward, the mandrels holding the graft against the deformation portion of the anvil. As such, the deformation portion of the anvil causes the open end of the graft to deform into the flange.

The present invention is a device that allows a surgeon to form a flange of appropriate size and shape into the end of a vascular graft while in the operating room, during a surgical procedure. The present device allows the surgeon to form the proper flange in the open end of a vascular graft cut to the appropriate length, whether the graft is made from synthetic or natural materials, and is a tool that can be operated with one hand while holding the graft with the other hand. The present invention allows various shaped flanges to be selected, and the size of such flanges can be readily controlled by the surgeon. The present device can be either autoclavable or disposable. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is an exploded perspective view of a hand tool of the present invention;

FIG. 2 is a cross-sectional, partially exploded view of the invention, illustrating the hand tool forming a flange onto the end of a vascular graft;

FIG. 3 is a partial perspective view of the end of the vascular graft before the flange is formed thereon with the hand tool; and

FIG. 4 is a cross-sectional, partially exploded view of the invention, illustrating a trigger, anvil and mandrels of the invention in more detail.

### DETAILED DESCRIPTION OF THE PREFERRED EMBODIMENT

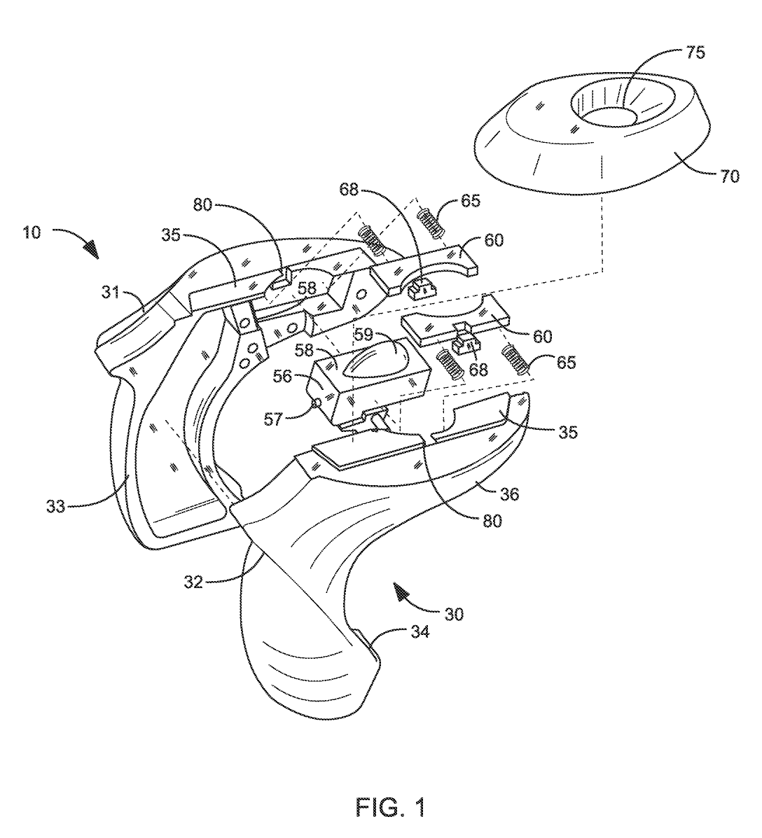

FIG. 1 illustrates a hand tool 10 for forming a flange 15 on the end 25 of a vascular graft 20. FIG. 3 illustrates the end 25 of the vascular graft 20 before the flange 15 is formed therein. A user of such a device may grasp the hand tool with one hand (not shown), insert the graft 20 into the tool 10, and form the flange 15, all while in an operating environment for example.

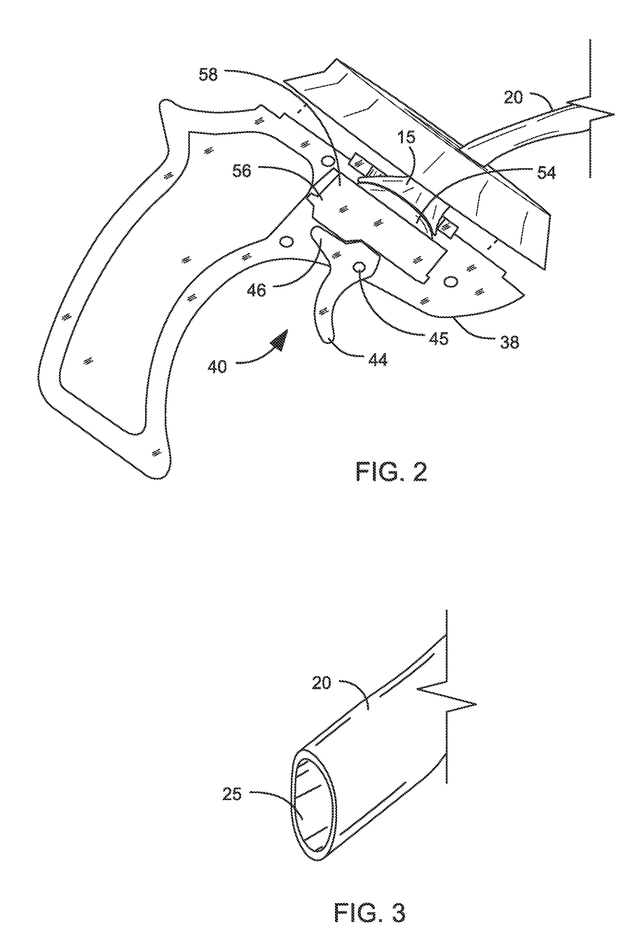

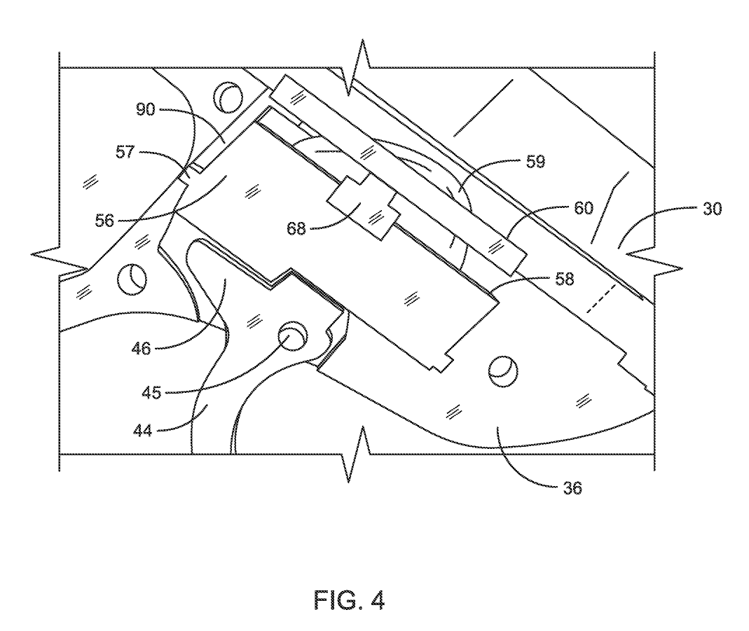

The hand tool 10 includes a housing 30 having a handle portion 34 fixed to a trigger portion 36. The handle portion 34 is adapted to be held by a human hand. A trigger 40 is pivotally fixed within the housing 30 at a lower side 38 of the trigger portion 36, and includes a finger portion 44 projecting downwardly from the housing 30 (FIGS. 2 and 4). The trigger 40 also includes an actuator portion 46, connected proximate a pivot 45 that is connected itself to the finger portion 44. As such, when the finger portion 44 is pulled towards the handle portion 34 of the housing 30, the trigger 40 pivots around the pivot 45 to cause the actuator portion 46 to lift within the housing 30. The finger portion 44 is adapted to be actuated by a forefinger of the human hand (not shown) while the hand is grasping the handle portion 34 of the housing 30, similarly to a convention pistol, for example.

The housing 30 is preferably made from a rigid plastic or metal material. In either case the housing 30 preferably comprises a left portion 31 and a right portion 32 (FIG. 1), each portion 31,32 fixed together along a joining center line 33 with ultrasonic welding, mechanical fasters, or the like (not shown).

An anvil 50 is pivotally fixed at a first end 54 thereof within and to the trigger portion 36 of the housing 30. Further, the anvil 50 is slidably fixed at a second end 56 to the actuator portion 46 of the trigger 40. The anvil 50 has a rigid top surface 58 with a deformation portion 59 thereon (FIG. 1). Preferably the housing 30 further includes a generally vertical guide slot 90 proximate to the second end 56 of the anvil 50, and the anvil 50 further includes a guide pin 57 extending rearwardly therefrom for slidable engagement with the vertical guide slot 90 of the housing 30. as such, the second end 56 of the anvil 50 is confined to vertical movement along the vertical guide slot 90 of the housing 30.

A plurality of mandrels 60 is adapted to laterally engage the deformation portion 59 of the anvil 50, and each include a spring means 65 for urging the mandrels 60 towards the deformation portion 59 of the anvil 50. The spring means 65 may be coil springs 65, as illustrated in FIG. 1, flat springs, resilient foam material, or the like (not shown). Preferably the trigger portion 36 of the housing 30 further includes a plurality of guide slots 80, and each mandrel 60 further includes a lateral guide pin 68 for engaging one of the slots 80, such that each mandrel 60 may only move laterally away from the anvil 50 along one of the slots 80 (FIGS. 1 and 4).

A guide collar 70 is fixed to an upper side 35 of the trigger portion 36 of the housing 30 and has an aperture 75 therein for receiving the open end 25 of the graft 20 (FIG. 3) and guiding same onto the deformation portion 59 of the anvil 30. With the graft 20 inserted into the aperture 75 of the guide collar 70 in such a fashion, the open end 25 of the graft 20 engages the deformation portion 59 of the anvil 50. At this point, manually pulling the finger portion 44 of the trigger 40 causes the actuator portion 46 of the trigger 40 to force the second end 56 of the anvil 50 upward, the mandrels 60 holding the graft 20 against the deformation portion 59 of the anvil 50. As such, the deformation portion 59 of the anvil 50 causes the open end 25 of the graft 20 to deform into the flange 15 (FIG. 2).

In one embodiment, the guide collar 70 can be readily removed, such as by sliding the collar 70 along the housing 30 to release it. As such, an alternate anvil 50 having a different deformation portion 59 shape may be readily substituted into the tool 10. alternately, a selection of such tools 10 may be available to the surgeon, each having a different shaped deformation portion 59 as may be required. The size of the flange 15 is controllable by the distance the trigger 40 is pulled, giving the surgeon complete control over the size of the resulting flange 15.

The hand tool 10 is preferably autoclavable, with all components thereof being able to withstand the heat generated by the autoclave processes. Alternately, the hand tool 10 may be made from relatively inexpensive rigid plastic materials, with the exception of the anvil 50 and mandrels 60 each being made form a relatively inexpensive aluminum or other metal material, such that the hand tool 10 is disposable after a few uses with the same patient.

While a particular form of the invention has been illustrated and described, it will be apparent that various modifications can be made without departing from the spirit and scope of the invention. For example, the exact placement number of mandrels 60 may be two, three, or four, if desired, or any other suitable number, provided that each mandrel 60 is urged by the spring means 65 towards the deformation portion 59 of the anvil 50 and can capture the end 25 of the vascular graft 20 therebetween while the trigger 40 is actuated. Accordingly, it is not intended that the invention be limited, except as by the appended claims.

What is claimed is:

1. A hand tool for forming a flange on a vascular graft, comprising:
a housing including a handle portion fixed to a trigger portion, the handle portion being adapted to be held by a human hand;
a trigger pivotally fixed within the housing at a lower side of the trigger portion thereof, the trigger including a finger portion projecting downwardly from the housing and an actuator portion, each mutually connected proximate a pivot;
an anvil pivotally fixed at a first end within and to the trigger portion of the housing and slidably fixed at a second end to the actuator portion of the trigger, the anvil having a rigid top surface with a deformation portion thereon;
a plurality of mandrels adapted to laterally engage the deformation portion of the anvil, the mandrels including spring means for urging the mandrels toward the deformation portion of the anvil; and
a guide collar fixed to an upper side of the trigger portion of the housing, the guide collar having an aperture therein for receiving the graft and guiding it onto the deformation portion of the anvil;
whereby with the graft inserted into the aperture of the guide collar, an open end of the graft engaging the deformation portion of the anvil, the trigger may be pulled rearward to cause the actuator portion to force the second end of the anvil upward, the mandrels holding the graft against the deformation portion of the anvil, such that the deformation portion of the anvil causes the open end of the graft to deform into the flange.

2. The hand tool of claim 1 wherein the trigger portion of the housing further includes a plurality of guide slots and each mandrel further includes a lateral guide pin for engaging one of the slots, whereby each mandrel may move laterally away from the anvil along one of the slots.

3. The hand tool of claim 1 wherein the trigger may be actuated by a forefinger of the human hand while the hand is grasping the handle.

4. The hand tool of claim 1 wherein the housing comprises a left portion and a right portion, each portion fixed along a joining center line.

5. The hand tool of claim 1 wherein the housing is made from a rigid plastic material.

6. The hand tool of claim 1 wherein the housing is made from a rigid metallic material.

7. The hand tool of claim 1 wherein the housing, guiding collar, mandrels, trigger portion, and anvil are each made from an autoclavable material.

8. The hand tool of claim 1 wherein the housing further includes a generally vertical guide slot proximate the second end of the anvil, and wherein the anvil further includes a guide pin extending rearwardly therefrom for slidable engagement with the vertical guide slot of the housing, whereby the second end of the anvil is confined to vertical movement along the vertical guide slot of the housing.

9. The hand tool of claim 1 wherein the guide collar is manually detachable from the housing and the anvil is removable, whereby the anvil may be replaced with an alternate anvil having a differently shaped deformation portion as may be required.

10. A method of forming a flange in the open end of a vascular graft, comprising the steps of:
a) providing a hand tool as recited in claim 1;
b) inserting the open end of the vascular graft into the hand tool against a deformation portion of an anvil thereof;
c) manually pulling a trigger of the hand tool to force the deformation portion of the anvil into the open end of the vascular graft;
d) releasing the trigger when the flange formed in the vascular graft is the correct size; and
e) removing the vascular graft from the tool.
